(19) United States
(12) Patent Application Publication
(10) Pub. No.: US 2009/0023170 A1
(43) Pub. Date: Jan. 22, 2009

(54) Methods And Kits For The Detection of Biotoxic and Antibiotic Residues

(75) Inventor: Nathan Citri, Jerusalem (IL)

Correspondence Address: BAKER BOTTS LLP;C/O INTELLECTUAL PROPERTY DEPARTMENT, THE WARNER, SUITE 1300, 1299 PENNSYLVANIA AVE, NW, WASHINGTON, DC, 20004-2400, US

(73) Assignee: RAVGALAI LIMITED, Ness Ziona (IL)

(21) Appl. No.: 11/574,005

(22) PCT Filed: Aug. 25, 2005
(86) PCT No.: PCT/IL2005/000922

§ 371 Date: Feb. 6, 2008

(30) Foreign Application Priority Data
Aug. 31, 2004 (IL) 163821
Jul. 18, 2005 (IL) 169721

(51) Int. Cl. C12Q 1/18; C12Q 1/34

(52) U.S. Cl. 435/18; 435/32

(57) ABSTRACT

The invention provides a kit for the detection of antibiotic residues comprising inducible bacterial spores which produce an induced enzyme after germination and induction; a germinant that triggers rapid germination of said spores; an inducer that triggers the production of the specific enzyme of interest; a substrate upon which said enzyme acts; and a detector of the activity of said enzyme on said substrate.

The present invention relates to methods and kits for the detection of biotoxic and antibiotic residues. More particularly, the present invention relates to methods and kits fit for visual and automated interpretation for the detection of antibiotic residues and toxic materials in milk, water, food and other consumables.

There is a need for a simple, rapid, single test for the detection of antibiotic residues in milk and in various stages of milk and food production.

Two basic approaches are currently applied. One approach aims at detecting a predetermined residue with the aid of specific sensors. The sensor is typically a specific antibody or reporter to which said residue would bind. There are numerous ways of detecting such binding and we may refer to the corresponding methods as SAM (specific assay methods).

The main advantage of SAM is speed: a SAM based test can be completed in minutes. The main disadvantage is the narrow specificity: a SAM based test will detect the residue that it was designed to detect and nothing else. Each residue requires a different sensor and it is unrealistic to expect that any commercially viable test based on this approach will provide an answer to the question whether a specimen is free of antibiotics.

By contrast, that question can be answered by methods based on the alternative approach, namely that a microbiological assay (MAS) will detect the biological effect of the antibiotics on a test organism. With that approach it is possible to ask whether the specimen contains any antibacterial substance and that, by definition, includes the various antibiotics. Hence an appropriate choice of a test organism will allow detection of the entire range of antibiotics, which are relevant, and to use a single assay for detecting any contaminant to which such organism is susceptible.

MAS based tests are very popular but relatively slow. The fastest and most widely used test requires hours of incubation. This is very unsatisfactory when decision has to be made as soon as possible and any delay is likely to be very costly.

In the present invention we combine the MAS approach with the speed of the SAM procedures.

The biological principles of the present invention are those applied in two earlier patents by the present inventor (U.S. Pat. No. 4,381,343 and U.S. Pat. No. 5,614,375). Briefly these principles are as follows:
1. A penicillinase producing bacterial strain is used as a sensor of inhibitors (antibiotics and generally biotoxic contaminants).
2. Conditions favorable to the initiation of penicillinase production are provided.
3. The level of penicillinase produced under such conditions in the presence of the sample tested is compared with that of the contaminant-free control.
4. In preferred embodiments the comparison is based on decolorization of a blue-black starch-iodine complex by penicilloic acid which is the product of the enzyme penicillinase acting on its substrate, penicillin.

In the first of the two patents use was made of log phase cultures of inducible penicillinase producers so as to be able to construct kits that could detect inhibitors (as in paragraph 1, above) and kits for detecting penicillin and other beta-lactam antibiotics that can be detected as inducers rather than inhibitors of penicillinase formation by such strains.

A major drawback of that system was that it could not be adapted to provide dry, stable and robust kits since only freshly grown log cultures were found to respond to the presence of inducers.

In the second of said patents, use is made of washed, activated spores that are essentially free of preformed penicillinase. The latter is formed upon germination of spores of a constitutive penicillinase producing bacillus. That was the basis of dry, robust kits designed for detection of biotoxic contaminants but obviously unsuitable for detection of penicillin and other beta-lactam antibiotics.

According to the present invention there is now provided a kit for the detection of antibiotic residues comprising;
One) inducible bacterial spores which produce an induced enzyme after germination and induction;
Two) a germinant that triggers rapid germination of said spores;
Three) an inducer that triggers the production of the specific enzyme of interest;
Four) a substrate upon which said enzyme acts; and
Five) a detector of the activity of said enzyme on said substrate.

In another aspect of the present invention there is now provided a method for the detection of antibiotic residues comprising
One) Placing a sample and a control specimen in separate test tubes for incubation.
Two) Placing said test tubes in an incubator at about 35° C.
Three) Adding a substrate both to said sample-containing test tube and said control-containing test tube in said incubator.
Four) Adding an indicator solution to said sample-containing test tube and to said control-containing test tube in said incubator; and
Five) Noting results in both test tubes.

As will be evident from the following description and examples the present invention applies those principles in ways that are novel both in concept and in technology enabling construction of kits that combine the desired advantages, while avoiding the limitations, mentioned above Such kits will:
a. Detect both beta-lactam and non-beta-lactam antibiotic as well as other toxic compounds in a single test;
b. Consist of dry and stable reagents, robust and storable at ambient temperatures;
c. Require no preparation, act faster and be simpler to use than previously available kits.

The differences between the present invention and those in previously mentioned patents are summarized in Table 1.

TABLE 1

| | U.S. Pat. No. | U.S. Pat. No. | Present |
|---|---|---|---|
| | 4,381,343 | 5,614,375 | invention |
| Test system | Log phase | Constitutive spores | Inducible |
| | bacterial culture | | spores |
| Preparation for | Seeding: 2 stages | None | None |
| testing | | | |
| Kits | Wet | Dry | Dry |
| Induction | Separate step | Not applicable | Built-in |
| Substrate | Separate step | Not applicable | Built-in or |
| addition | | | separate step |
| Germination | Not applicable | Triggers test | Triggers test |
| Antibiotics | Either BLA^{x} | Only non-BLA^{xx} | Both BLA^{x} and |
| detected | or non-BLA^{xx} | | non-BLA^{xx} |
| Manual steps | 6-7 | 2 | 1-2 |
| Total testing | >120 min. | <30 to <60 min. | 20-25 min. |
| time | | | |

^{x}BLA = β-lactam antibiotic
^{xx}non-BLA = non β-lactam antibiotic

The present system is designed so as to enable combination of the two approaches used for the detection of BLA and non-BLA respectively in a single testing procedure. It must be emphasized that such combination is anything but obvious since the two approaches are inherently contradictory. Whereas non-BLA are detected as inhibitors of penicillinase formation under conditions where, in their absence, said enzyme would be formed, BLA are detected by their ability to induce penicillinase formation under conditions where, in their absence, said enzyme would not be formed. What is more, the BLA detecting system incorporates, as its main reagent, a massive amount of BLA in the form of penicillin, the substrate of the enzymatic reaction catalyzed by penicillinase.

In the present invention the contradictory requirements reviewed above are all met by designs based on the innovative concept that a set of correctly timed sequential steps can separate the incompatible interactions and provide a solution in the form of a single testing system.

### Properties of the System

The system makes use of an enzyme, to serve as an indicator of the presence of [a] an inducer, which is necessary for its production and [b] an inhibitor, which prevents its production even in the presence of the inducer.

The system consists of the following elements:
1. Inducible bacterial spores which, by definition, can produce an induced enzyme only after germination and induction.
2. A germinant that triggers rapid germination of above spores.
3. An inducer that triggers the production of the specific enzyme of interest.
4. A substrate upon which said enzyme acts.
5. A detector of the activity of said enzyme on said substrate.

In the specific case presented here the enzyme is penicillinase and penicillin is both the inducer and the substrate of said enzyme. The specific detector here is a blue-black solution of starch-iodide-iodine or an absorbent strip impregnated with the detector solution.

All five elements are stored dry and can be combined and stored as ready-made kits since no interaction will take place in the dry state. In all kits, however, penicillin must be initially physically separated from the other components. The system is activated upon the addition of the liquid sample to be tested. The sequence of events that are triggered [and accelerated at 30° C.-40° C.] is as follows:
1. The germinant is dissolved and the spores germinate and respond to the contents of the liquid sample
   i. If the sample contains penicillin or any other BLA, penicillinase production is induced.
   ii. If the sample contains an inhibitor such as non-BLA, penicillinase production is inhibited.
   iii. A control identical to the test sample but known to contain neither inducer nor inhibitor must be included.
2. After a brief incubation [see Examples] penicillin is released into the reaction. The release can be fully automatic or partly manual. The initial contact introduces penicillin as the inducer whereas subsequently penicillin serves as the substrate required for saturation, and hence for maximal velocity, for any induced enzyme activity.
3. Production of penicillinase can be monitored by detecting the product of the catalytic reaction, penicilloic acid, which unlike the intact substrate, penicillin, removes iodine from its complex with starch. This will result in decolorization of the detector in the solution, or creation of white areas on the nearly black detector strip on contact with a solution containing penicilloic acid.
4. Results of the test are based on comparing the decolorization steps in the test sample and in the control. Comparison can be carried out electro-optically or manually (visually), as illustrated in the Examples below.
5. Interpretation of Results:
   i. Control provides the level of penicillinase expected after the addition of the inducer in mid-test [see {2} above]
   ii. A higher level in the test sample means that penicillinase production, was started before addition of penicillin and hence must have been induced by BLA contaminating the sample
   iii. A level lower than that of the control results from partial inhibition by traces of an inhibitor [e.g., non-BLA] contaminating the sample. With heavier contamination no penicillinase will be produced.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### SPECIFIC EXAMPLES

### Example 1

### A. Definitions

1. Sample is an aliquot of a liquid specimen, such as milk or blood or of the liquid in which a solid specimen is collected and stored.
2. Test kit is a test tube where the sample and control specimens are delivered (each to a separate tube), and upon arrival, initiates one or more biological or chemical reactions. The bottom of each test tube is lined with dry reagents consisting of a mixture of germinants and of spores of an inducible penicillinase producing bacterial strain.
3. Robot, pipette or a dispenser is a device for transporting the sample to the test kit and/or transporting substrate and/or indicator solution to test kit during test.
4. Interpreter is any electro-optical device connected to an electronic processor (PC for example) capable of image processing, recording and saving test results. Visual interpretation of test results is always possible.

### B. Test Procedure and Results

1. Samples and control (0.25 m/l of each) are placed simultaneously in the respective test tubes for incubation at 37° C.
2. At 15 minutes the beta-lactam is released or added to sample and control tubes to act as an inducer and, subsequently, as the substrate for the induced enzyme.
3. At 20 minutes indicator solution is released or added to sample and control tubes.
4. Interpretation is then carried out by series of digital photos or visual observation.
   The rate of the decolorization of the indicator by the sample is compared to that of the control.

Sample faster when contaminated by BLA.

Sample slower when contaminated by non-BLA.

Sample equal to control when free of any detectable antibacterial activity.

### Example 2

### A. Definitions

1. Sample is an aliquot of a liquid specimen, such as described in example 1 above.
2. Station is the site where the sample is delivered or conveyed [see below] and, upon arrival, initiates one or more biological or chemical reactions.
3. Conveyor is a device for transporting the sample from one station to another by capillary action. The transport is mediated by cotton wool or by a strip of absorbing material, such as filter paper, or by a wick of any inert composition or by any combination of the above.
   B. Properties of the Conveyor In its simplest version the conveyor consists of a double-headed cotton wool swab with an absorbent strip connecting the heads.
1. In this Example the shape and size of the conveyor is designed to fit into a 55×11 mm test tube. The bottom of the tube is lined with dry spores and reagents and serves as Station A to which the Sample is delivered.
2. Stations B-D are incorporated in the Conveyor. The lower cotton wool head that may be impregnated with a reagent serves as Station B.
3. The absorbent strip is impregnated with an indicator dye and serves as Station C to which the Sample is transferred from Station B by the wicking action of said strip.
4. The strip, which connects the 2 ends of the Conveyor, now delivers the Sample to the upper cotton wool head that is impregnated with another reagent and thus serves as Station D.
5. The time spent at each Station may be crucial for the testing procedure. The Conveyor can be designed to automatically control the timing of arrival and departure of the Sample. This can be done in several ways as for example by adjusting the distance between the Stations or impeding the absorption or the flow of the Sample as needed.
6. The Conveyor can be designed to slow down the flow [see previous paragraph] without interfering with diffusion of small molecules such as substrates and products of a many enzymatic reactions [see also Specific Examples below]. This can be achieved by using inert gels [e.g., agar] to impregnate contact areas in or along the strip and create similar barriers to delay flow but allow free diffusion.

### Example 3

### Detection of Antibiotic Residues in Milk: a Single-Step Test Kit.

In this illustrative Example the test tube and conveyor configurations are as described above.
Test Tubes: The bottom of each test tube [Station A] is lined with dry reagents consisting of a mixture of germinant and of spores of an inducible penicillinase producing bacterial strain.
Sample: 0.5 mL of milk to be tested
Control: 0.5 mL of antibiotic free milk
Conveyor: Lower head is impregnated with a pH indicator [Station B]. Upper end is impregnated with penicillin [Station D]. Strip conveying Sample from Station B to D is impregnated with a blue-black starch-iodine-iodide solution.

### Testing

### 1. Setting Up Test:

Place Sample and Control in above test tubes add a Conveyor swab to each and incubate at 37° C. for 25 min.

### 2 Reading Results:

Observe levels of deccolorization of Sample and Control strips on respective Conveyors.

If Sample level is above Control level, Sample shows contamination with BLA [beta lactam antibiotics].

If Sample level is below Control level, Sample contains other [non-BLA] contaminants.

If Sample and Control levels are similar, Sample shows no detectable residues of antibiotics.

### Comments

1. Placing liquid sample in test tube [Station A] activates the system: the spores germinate and respond immediately to any antibiotic present in the sample that may induce or inhibit penicillinase formation.
2. Immersion of the Conveyor in said liquid and consequent wicking action of the strip starts the transfer of reactants from Station A to Station D.
3. The rate of transfer in this Example was set so as to reach Station D in 11 min. At that point gradual diffusion of penicillin is initiated and, within the following 4 min. induction takes place unless an inhibitor is present.

### Example 4

### Detection of Antibiotic Residues in Milk: a Self-Recording Kit.

In this Example the test tubes and the Sample and Control are as above. The Conveyor is, however, replaced with 2 elements:
i. A test stick, a swab impregnated with penicillin and serving to deliver the inducer and substrate and to check progress [see below].
ii. A test card, a card impregnated with a blue-black solution of starch-iodine-iodide.

### Testing:

1. Place Sample and Control in the test tubes for incubation at 37° C. Start timer.
2. At 15 min. add a test stick to each test tube.
3. At 20 min. check progress by using each test stick to wet a marked spot on test card.
4. Observe changes taking place in wetted spots.

### Results

If the spots of Sample and Control decolorized at the same rate, Sample is shown to be free of detectable antibiotic residues.

If Sample faster—BLA present, whereas

If Control faster—non-BLA present, in the milk tested.

### Record

The air-dried test card provides a storable record of the results.

### Comments

1. The test stick in this Example serves three distinct functions, namely:
   Delivery of inducer
   Delivery of substrate
   A sampling device for spot testing on test card
2. Delivery of inducer as distinct from delivery of substrate is enabled by the gradual release of penicillin due to the design of the penicillin-impregnated head of the test stick.

### Example 5

### Test Kit According to the Present Invention

The test kit set consists of two member elements detecting the presence of antibiotic residues in the tested specimen. One member is more sensitive to antibiotics of Beta Lactam group (hereinafter BL) and the second member is more sensitive to antibiotics belonging to other groups that are not Beta Lactam (hereinafter NBL). Each test kit consists of two reaction test tubes A and B:
The sample is an aliquot of a liquid specimen, such as milk or blood or of the liquid in which a solid specimen is collected and stored.
1. The BL is a bio-reaction tube in which its bottom is lined with lyophilized reagents consisting of a mixture of quartz grains, germinant and spores of an inducible penicillinase producing bacterial strain. Into test kit bio-reaction tube BL, the sample and control specimens are delivered (each to a separate tube), and upon arrival, initiate one or more biological or chemical reactions.
2. The NBL is a bio-reaction tube in which its bottom is lined with lyophilized reagents consisting of a mixture of quartz grains, germinant, beta lactam and spores of an inducible penicillinase producing bacterial strain. Into test kit bio-reaction tube NBL the sample and control specimens are delivered (each to a separate tube), and upon arrival, initiate one or more biological or chemical reactions.
3. The reaction tubes A and B are enzyme substrate chromogen interaction tubes in which the bottom of each test tube is lined with dried beta lactam as substrate reagents.
   3.1. “Processed” aliquots of sample and control from each BL tubes in tests are placed in separate A tubes and external liquid (Starch-Iodine-iodide complex) chromogen-indicator reagent is also delivered to same A tubes initiating one or more biochemical reactions.
   3.2. “Processed” aliquots of sample and control from each NBL tubes in tests are placed in separate B tubes and external liquid (Starch-Iodine-iodide complex) chromogen-indicator reagent is also delivered to same B tubes initiating one or more biochemical reactions.
   3.3. Another option is that each A and/or B tubes will contain the Starch-Iodine-iodide complex chromogen-indicator reagent in a dry form (for example lyophilized).
4. Liquid Handling Accessories: Robot, pipette or a dispenser is a device for transporting the sample to the test kit and transporting “processed” aliquots of sample and control from both tubes BL and NBL to A and B tubes respectfully and/or deliver the external liquid chromogen-indicator reagent to A and B tubes initiating one or more biochemical reactions, in the relevant test kit devices during test.
5. Interpretation Unit: Interpreter is an electro optical device (CD camera) connected to an electronic processor (PC for example) capable of image processing, recording and saving test results. Visual observation of test results is as well an alternative option.
6. Shaker Incubator: A close cabin apparatus in which all assay stages are taken place. Two main functions are kept stable and constant within the Shaker Incubator; 1. Temperature; 37° C., 2. Fixed shaking speed (RPM) in a constant rotation radius. The electro optical device (CD camera) and illuminating device are placed within shaker incubator. Photographs of A and B tubes contents are taken at desired intervals and images are delivered on line to interpreting device (data processor) for interpreting test results.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. A kit for the detection of antibiotic residues comprising:
a) inducible bacterial spores which produce an induced enzyme after germination and induction;
b) a germinant that triggers rapid germination of said spores;
c) an inducer that triggers the production of the specific enzyme of interest;
d) a substrate upon which said enzyme acts; and
e) a detector of the activity of said enzyme on said substrate.

2. A kit according to claim 1, wherein said enzyme is penicillinase and penicillin is both the inducer and the substrate of said enzyme.

3. A kit according to claim 1, wherein said detector is a blue-black solution of starch-iodide-iodine.

4. A kit according to claim 1, wherein said detector is an absorbent strip impregnated with a blue-black solution of starch-iodide-iodine.

5. A kit according to claim 1, wherein all of the elements a, b, c, and d of said kit are stored in a dry state.

6. A kit according to claim 1, wherein said detector is stored in a liquid state and added during the test.

7. A kit according to claim 2, wherein said penicillin is physically separated from said other components by coating.

8. A kit according to claim 1 further comprising inert mechanical mixing means for combination with an analyte solution to physically facilitate the homogenization thereof.

9. A kit according to claim 7 wherein said inert mechanical mixing means comprising borosilicate glass-spheres.

10. A method for the detection of antibiotic residues comprising
a) Placing a sample and a control specimen in separate kit tubes for incubation.
b) Placing said test tubes in an incubator at about 37° C.
c) Adding a substrate both to said sample-containing test tube and said control-containing test tube in said incubator.
d) Adding an indicator solution to said sample-containing test tube and said control-containing test tube in said incubator; and
e) Noting results in both test tubes.

11. A method according to claim 9 wherein said substrate is added in step c) at a period of about 10-20 minutes after the addition of said sample to said kit tube for incubation.

12. A method according to claim 10 wherein said indicator solution is added in step d) to said sample at a period of about 15-25 minutes after the addition of said substrate to said kit tube.
